(19) United States
(12) Patent Application Publication
(10) Pub. No.: US 2010/0324378 A1
(43) Pub. Date: Dec. 23, 2010

(54) PHYSIOLOGIC SIGNAL MONITORING USING ULTRASOUND SIGNALS FROM IMPLANTED DEVICES

(76) Inventors: Binh C. Tran, Minneapolis, MN (US); Jon Peterson, Mahtomedi, MN (US); John D. Hatlestad, Maplewood, MN (US); Frank Ingle, Palo Alto, CA (US)

Correspondence Address: FAEGRE & BENSON LLP;PATENT DOCKETING - INTELLECTUAL PROPERTY (32469), 2200 WELLS FARGO CENTER, 90 SOUTH SEVENTH STREET, MINNEAPOLIS, MN, 55402-3901, US

(21) Appl. No.: 12/782,369

(22) Filed: May 18, 2010

Related U.S. Application Data
(60) Provisional application No. 61/187,817, filed on Jun. 17, 2009.

(51) Int. Cl. A61B 5/0215; A61B 5/00; A61B 5/02; A61B 5/08

(52) U.S. Cl. 600/301; 600/300; 600/486; 600/508; 600/529

(57) ABSTRACT

Devices, systems, and methods for monitoring and analyzing physiologic parameters within the body using an intrabody ultrasound signal are disclosed. An illustrative method includes receiving an ultrasound signal transmitted from a remote device containing encoded sensor data, converting the ultrasound signal into an electrical signal, decoding the sensor data from the electrical signal and generating a first physiological waveform, generating a second physiological waveform by analyzing fluctuations of the electrical signal caused by physiologic modulation of the ultrasound signal during propagation through the body, and analyzing one or more characteristics of the first and second waveforms to determine one or more physiologic parameters within the body.

### CROSS-REFERENCE TO RELATED APPLICATIONS

This application claims benefit under 35 U.S.C. §119 to U.S. Provisional Application No. 61/187,817, filed Jun. 17, 2009, entitled “Physiologic Signal Monitoring Using Ultrasound Signals From Implanted Devices,” which is incorporated herein by reference in its entirety for all purposes.

### TECHNICAL FIELD

The present invention relates generally to the monitoring of physiologic parameters within the body. More specifically, the present invention relates to devices, systems, and methods for monitoring and analyzing physiologic parameters within the body using intrabody ultrasound signals.

### BACKGROUND

Implantable medical devices (IMDs) are utilized in a variety of medical applications for sensing and deriving physiologic parameters within the body. In cardiac rhythm management (CRM) systems used to monitor the status of a patient's heart, for example, an implantable sensor can be configured to sense various physiologic parameters occurring in the atria and/or ventricles of the heart, or in the vessels leading into or from the heart. The sensor data obtained from such devices can be used to derive various hemodynamic parameters such as heart rate, cardiac output, and stroke volume. In one such system, for example, a pressure sensor implanted within a pulmonary artery can be used to sense blood pressure, which can then be used by the pressure sensor or another device located inside or outside of the body to determine end diastolic pressure (EDP). The pressure waveform and EDP can be transmitted to another implanted or external device and used by a physician in the long term management of patients with heart failure. In some cases, an implantable device such as a pacemaker, defibrillator, or cardiac resynchronization device can deliver a therapy to the patient based in part on the pressure readings taken by the pressure sensor.

### SUMMARY

The present invention relates to devices, systems, and methods for monitoring and analyzing physiologic parameters within the body using intrabody ultrasound signals.

In Example 1, a method for determining one or more time-varying physiologic parameters within the body of a patient using intrabody ultrasound signals includes receiving an ultrasound signal transmitted from a remote device located within the body, the ultrasound signal including encoded sensor data measured by the remote device; transducing the ultrasound signal into an electrical signal; decoding the sensor data from the electrical signal and generating a first physiological waveform corresponding to the sensor data measured by the remote device; and generating a second physiological waveform by analyzing fluctuations of the electrical signal caused by physiologic modulation of the ultrasound signal during propagation through the body.

In Example 2, the method according to Example 1, further including analyzing at least one characteristic of the first and second physiological waveforms to determine one or more physiological parameters within the body.

In Example 3, the method according to any of Examples 1-2, wherein the first physiological waveform is a pressure waveform.

In Example 4, the method according to any of Examples 1-3, wherein the second physiological waveform is a respiration waveform.

In Example 5, the method according to any of Examples 1-4, wherein the second physiological waveform is a cardiac waveform.

In Example 6, the method according to any of Examples 1-5, further comprising using the one or more physiologic parameters to calibrate another device within the body.

In Example 7, the method according to any of Examples 1-6, wherein the remote device is a pressure sensor implanted within a pulmonary artery, and wherein the encoded sensor data comprises blood pressure data measured by the remote device within the pulmonary artery.

In Example 8, the method according to any of Examples 1-7, wherein generating a second physiological waveform includes filtering the electrical signal with a low-pass or band-pass filter having a bandwidth corresponding to the frequency range of a physiologic signal of interest.

In Example 9, the method according to Example 2, wherein analyzing at least one characteristic of the first and second physiological waveforms to determine one or more physiologic parameters within the body includes detecting one or more peaks in the electrical signal and correlating the amplitude and timing of the peaks in the electrical signal with the measured sensor data from the first physiological waveform.

In Example 10, the method according to Example 2, wherein analyzing at least one characteristic of the first and second physiological waveforms includes determining the end expiration stage of the patient's respiration cycle.

In Example 11, the method according to Example 2, wherein analyzing at least one characteristic of the first and second physiological waveforms includes determining a respiration rate of the patient's respiration cycle.

In Example 12, the method according to Example 2, wherein analyzing at least one characteristic of the first and second physiological waveforms includes determining a tidal volume of the patient's respiration cycle.

In Example 13, the method according to Example 2, wherein analyzing at least one characteristic of the first and second physiological waveforms includes determining a heart rate.

In Example 14, the method according to Example 2, wherein analyzing at least one characteristic of the first and second physiological waveforms includes determining the presence of at least one of a cardiac arrhythmia, extra beat or skipped beat, or aperiodic cardiac event.

In Example 15, the method according to any of Examples 1-14, further comprising adjusting at least one operating parameter of the remote device in response to the one or more physiologic parameters.

In Example 16, the method according to any of Examples 1-15, further comprising determining one or more device-related parameters of the remote device based at least in part on the amplitude, phase, and/or time delay of a carrier signal component of the received ultrasound signal.

In Example 17, the method according to Example 16, wherein determining one or more device-related parameters of the remote device includes measuring a Doppler shift in the received ultrasonic signal.

In Example 18, the method according to Example 16, further comprising prompting the remote device to transmit a first ultrasound signal at a first frequency and a second ultrasonic signal at a second frequency different than the first frequency, and wherein determining one or more device-related parameters includes measuring a separation distance between the remote device and a communicating device in acoustic communication with the remote device based on a measured change in attenuation of the first and second ultrasound signals received by the communicating device.

In Example 19, a method for determining one or more time-varying physiologic parameters within the body of a patient using intrabody ultrasound signals includes transmitting an ultrasound signal from a remote device located within the body to a communicating device in acoustic communication with the remote device; receiving the ultrasound signal on an ultrasonic transducer of the communicating device and transducing the ultrasound signal into an electrical signal; generating a physiological waveform by analyzing fluctuations of the electrical signal caused by physiologic modulation of the ultrasound signal during propagation through the body; and analyzing the physiological waveform to determine one or more physiologic parameters within the body.

In Example 20, a system for determining one or more physiologic parameters within the body of a patient using an intrabody ultrasound signal includes a remote device including at least one ultrasound transducer adapted to transmit an intrabody ultrasound signal; a communicating device in acoustic communication with the remote device, the communicating device including at least one ultrasound transducer configured to receive the ultrasound signal and transduce the ultrasound signal into an electrical signal; and processing means for: generating a physiological waveform by analyzing fluctuations of the electrical signal caused by physiologic modulation of the ultrasound signal during propagation through the body, and analyzing at least one characteristic of the physiologic waveform to determine one or more physiologic parameters within the body.

In Example 21, the system according to Example 20, wherein the physiological waveform is a respiration waveform.

In Example 22, the system according to any of Examples 20-21, wherein the physiological waveform is a cardiac waveform.

In Example 23, the system according to any of Examples 20-22, wherein the remote device is configured to measure blood pressure within a vessel of the body.

In Example 24, the system according to Example 23, wherein the ultrasound signal includes encoded pressure data measured by the remote device, and wherein the processing means is further configured for decoding the pressure data from the ultrasound signal and generating a pressure wave corresponding to the pressure data measured by the remote device.

In Example 25, the system according to any of Examples 20-24, wherein the processing means is further configured for analyzing at least one characteristic of the physiologic waveform and at least one characteristic of the pressure waveform to determine one or more physiologic parameters within the body.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic view of an illustrative system employing a remote implantable medical device located within the body of a patient;

FIG. 2 is a block diagram showing several illustrative components of the remote implantable medical device of FIG. 1;

FIG. 3 is a block diagram showing several illustrative components of the external monitor of FIG. 1;

FIG. 4 is a block diagram showing several illustrative components of the ultrasound enabled pulse generator of FIG. 1;

FIG. 5 is a diagram showing several illustrative steps for sensing, sampling, encoding, and communicating a single pressure measurement through the body using the system of FIG. 1;

FIGS. 6A-6B are illustrative graphs showing the generation of a pressure waveform based on encoded sensor data taken by the remote implantable medical device and transmitted acoustically to a communicating device such as the external monitor and/or pulse generator of FIG. 1;

FIG. 7 is a graph showing the estimation of end diastolic pressure at expiration based on pulmonary artery pressure waveform data obtained from a remote implantable medical device implanted within a pulmonary artery;

FIG. 8 is a flow chart showing an illustrative method for determining one or more physiologic parameters within the body of a patient by analyzing the signal characteristics of an intrabody ultrasound signal;

FIGS. 9A-9B show an illustrative respiration waveform generated from an ultrasound signal transmitted through the body; and

FIGS. 10A-10B show the determination of end diastolic pressure at end expiration from an illustrative pressure waveform and corresponding respiration waveform of FIG. 9B.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

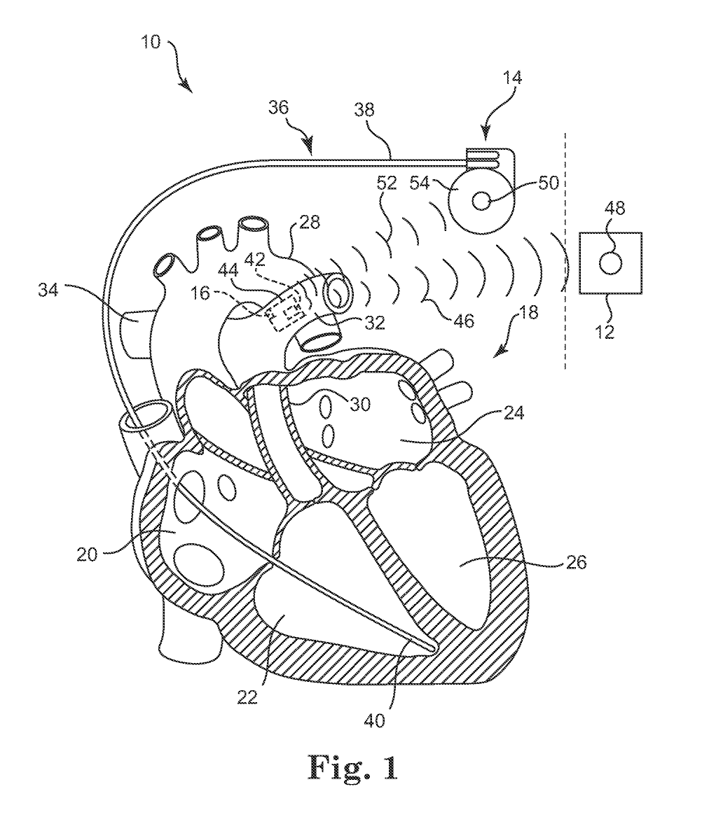

FIG. 1 is a schematic view of an illustrative system 10 employing a remote implantable medical device (IMD) located within the body of a patient. The system 10, illustratively a cardiac rhythm management system for providing cardiac rhythm management or cardiac disease management, includes an external monitor 12 (e.g., an external communicator, reader, or programmer), a pulse generator 14 implanted within the body, and at least one remote IMD 16 implanted deeply within the patient's body such as in one of the atria or ventricles of the patient's heart 18, or in one of the blood vessels leading into or from the heart 18. The heart 18 includes a right atrium 20, a right ventricle 22, a left atrium 24, a left ventricle 26, and an aorta 28. The right ventricle 22 leads to the main pulmonary artery 30 and the branches 32,34 of the main pulmonary artery 30.

In the illustrative system 10 depicted, the pulse generator 14 is coupled to a lead 36 deployed in the patient's heart 18. The pulse generator 14 can be implanted subcutaneously within the body, typically at a location such as in the patient's chest or abdomen, although other implantation locations are possible. A proximal portion 38 of the lead 36 can be coupled to or formed integrally with the pulse generator 14. A distal portion 40 of the lead 36, in turn, can be implanted at a desired location within the heart 18 such as the right ventricle 22, as shown. Although the illustrative system 10 depicts only a single lead 36 inserted into the patient's heart 18, in other embodiments the system 10 may include multiple leads so as to electrically stimulate other areas of the heart 18. In some embodiments, for example, the distal portion of a second lead (not shown) may be implanted in the right atrium 20. In addition, or in lieu, another lead may be implanted in the left side of the heart 18 (e.g., in the coronary veins) to stimulate the left side of the heart 18. Other types of leads such as epicardial leads may also be utilized in addition to, or in lieu of, the lead 36 depicted in FIG. 1.

During operation, the lead 36 is configured to convey electrical signals between the heart 18 and the pulse generator 14. For example, in those embodiments where the pulse generator 14 is a pacemaker, the lead 36 can be utilized to deliver electrical therapeutic stimulus for pacing the heart 18. In those embodiments where the pulse generator 14 is an implantable cardiac defibrillator, the lead 36 can be utilized to deliver electric shocks to the heart 18 in response to an event such as ventricular fibrillation. In some embodiments, the pulse generator 14 includes both pacing and defibrillation capabilities.

The remote IMD 16 can be configured to perform one or more designated functions, including the sensing of one or more physiologic parameters within the body. Example physiologic parameters that can be measured using the remote IMD 16 can include, but are not limited to, blood pressure, blood flow, and temperature. Various electrical, chemical, magnetic, and/or sound properties may also be sensed within the body via the remote IMD 16.

In the embodiment of FIG. 1, the remote IMD 16 comprises a pressure sensor implanted at a location deep within the body such as in the main pulmonary artery 30 or a branch 32,34 of the main pulmonary artery 30 (e.g., in the right or left pulmonary artery). An example of a pressure sensor suitable for use in sensing blood pressure in a pulmonary artery is described in U.S. Pat. No. 6,764,446, entitled “Implantable Pressure Sensors and Methods for Making and Using Them,” which is incorporated herein by reference in its entirety for all purposes. In use, the remote IMD 16 can be used to aid in the prediction of decompensation of a heart failure patient and/or to aid in optimizing cardiac resynchronization therapy via the pulse generator 14 by monitoring blood pressure within the body. In some embodiments, the remote IMD 16 can be configured to sense, detect, measure, calculate, and/or derive other associated parameters such as flow rate, maximum and minimum pressure, peak-to-peak pressure, rms pressure, and/or pressure rate change.

The remote IMD 16 may be implanted in other regions of the patient's vasculature, in other body lumens, or in other areas of the body, and may comprise any type of chronically implanted device adapted to deliver therapy and/or monitor biological and chemical parameters, properties, and functions. The remote IMD 16 can be tasked, either alone or with other implanted or external devices, to provide various therapies or diagnostics within the body. Although a single remote IMD 16 is depicted in FIG. 1, multiple such devices can be implanted at various locations within the body for sensing or monitoring physiologic parameters and/or providing therapy at multiple regions within the body.

An acoustic communication link may be established to permit wireless communications between the remote IMD 16 and the external monitor 12, between the remote IMD 16 and the pulse generator 14, and/or between the remote IMD 16 and one or more other devices located inside or outside of the body. In the illustrative system 10 of FIG. 1, for example, an ultrasonic transducer 42 disposed within the housing 44 of the remote IMD 16 is configured to transmit an ultrasound signal 46 towards the external monitor 12. An example ultrasonic transducer suitable for use with the remote IMD 16 for transmitting and receiving ultrasound signals is described in U.S. Pat. No. 6,140,740, entitled “Piezoelectric Transducer,” which is expressly incorporated herein by reference in its entirety for all purposes.

The external monitor 12 includes one or more ultrasonic transducers 48 configured to receive the ultrasound signal 46 and complete an acoustic link between the remote IMD 16 and the external monitor 12. In some cases, for example, the acoustic link established between the remote IMD 16 and the external monitor 12 can be used to wirelessly transmit sensor data, operational status information, and/or other information to the external monitor 12. An example telemetry system employing ultrasonic transducers is described in U.S. Pat. No. 7,024,248, entitled “Systems and Methods For Communicating With Implantable Devices,” which is incorporated herein by reference in its entirety for all purposes.

In some embodiments, the ultrasonic transducer(s) 48 for the external monitor 12 may transmit an ultrasound signal to the remote IMD 16 to prompt the IMD 16 to perform a desired operation. In one embodiment, for example, the external monitor 12 may transmit an acoustic wake-up command to the remote IMD 16, causing the IMD 16 to activate from an initial, low-power state for conserving power usage to an active, energized state for taking one or more sensor measurements and transmitting sensor data to the external monitor 12, to the pulse generator 14, and/or to another device located inside or outside of the body. In some embodiments, and as further discussed herein, the external monitor 12 may transmit an acoustic control signal that prompts the remote IMD 16 to wake up only a portion of the IMD 16 and transmit one or more ultrasonic pulses without activating the sensor circuitry within the IMD 16.

While the system 10 of FIG. 1 includes a remote IMD 16 that communicates with an external monitor 12, in other embodiments the remote IMD 16 communicates with other devices located inside or outside of the patient's body. As further shown in FIG. 1, for example, the remote IMD 16 may be in acoustic communication with the pulse generator 14, which can include one or more ultrasonic transducers 50 adapted to receive an ultrasound signal 52 transmitted by the remote IMD 16. In certain embodiments, the ultrasonic transducer(s) 50 are coupled to an interior portion of the can 54 that encloses the various components of the pulse generator 14. In other embodiments, the ultrasonic transducer(s) 50 are located outside of the can 54, on a header of the can 54, or are coupled to the pulse generator 14 through a feedthrough provided on the can 54.

Although the system 10 depicted in FIG. 1 shows an acoustic link between the remote IMD 16 and an external monitor 12, and/or between the IMD 16 and a pulse generator 14, in other embodiments an acoustic link can be established between the remote IMD 16 and another device implanted within the body. In some embodiments, for example, an acoustic link can be established between a primary IMD 16 and one or more secondary IMDs 16 implanted within the body.

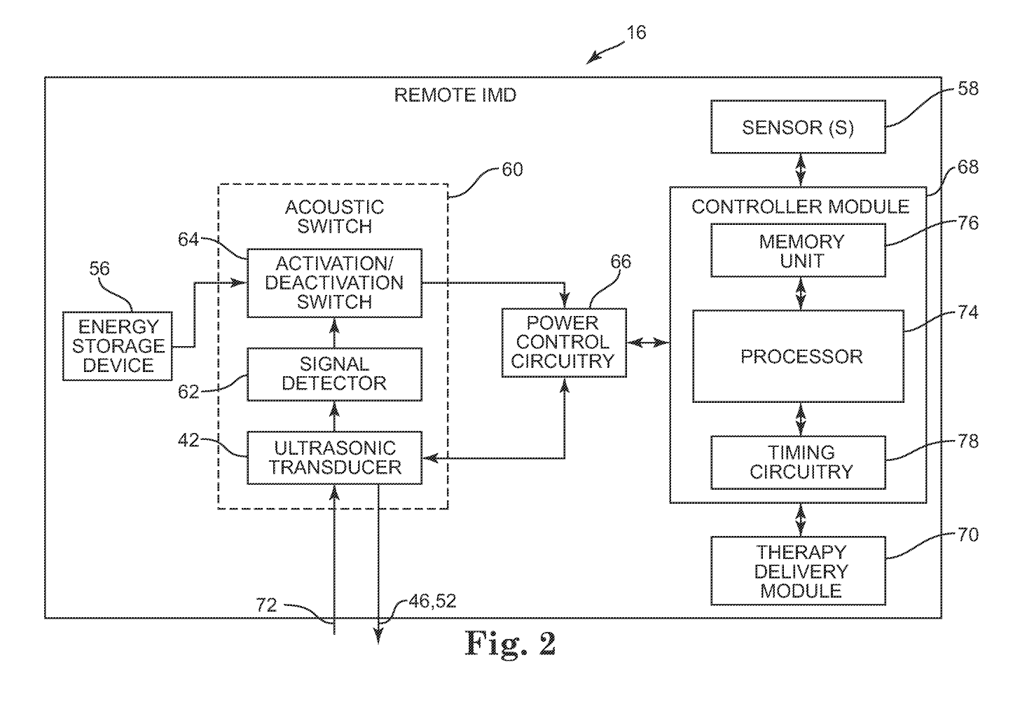

FIG. 2 is block diagram showing several illustrative components of the remote IMD 16 of FIG. 1. As shown in FIG. 2, the remote IMD 16 includes an energy storage device 56, a physiologic sensor 58, an acoustic switch 60 (including the acoustic transducer 42, a signal detector 62, and an activation/deactivation switch component 64), power control circuitry 66, and a controller module 68. The energy storage device 56 may be non-rechargeable or rechargeable, and supplies power to the physiologic sensor 58, the acoustic switch 60, the power control circuitry 66, and the controller module 68. The power control circuitry 66 is operatively connected to the acoustic switch 60, and is used to regulate the supply of power from the energy storage device 56 to the controller module 68.

The physiologic sensor 58 performs functions related to the sensing of one or more physiologic parameters within the body. In certain embodiments, for example, the physiologic sensor 58 comprises a pressure sensor adapted to measure blood pressure in the body. In one embodiment, the remote IMD 16 is implanted in a pulmonary artery of the patient, and the physiologic sensor 58 is adapted to sense blood pressure within the artery. In other embodiments, the physiologic sensor 58 is adapted to generate a signal related to other sensed physiologic parameters including, but not limited to, temperature, electrical impedance, pH, blood flow, and glucose level. In certain embodiments, the remote IMD 16 may also include a therapy delivery module 70 that performs one or more therapeutic functions (e.g., cardiac pacing or drug delivery) within the body in addition to, or in lieu of, the one or more sensing functions provided by the physiologic sensor 58.

The ultrasonic transducer 42 for the remote IMD 16 may include one or more piezoelectric transducer elements configured to transmit and receive ultrasound signals. In a reception mode of operation, the ultrasonic transducer 42 can be configured to receive a control signal 72 transmitted from the external monitor 12 and/or the pulse generator 14, which is fed to the controller module 68 when the remote IMD 16 is in an active state. In a transmit mode of operation, the ultrasonic transducer 42, or another ultrasonic transducer coupled to the remote IMD 16, is configured to transmit an ultrasound signal 46,52 to the external monitor 12, to the pulse generator 14, and/or to another device located inside or outside of the body. The transmitted ultrasound signal 46,52 can include sensor data obtained from the physiologic sensor 58, information relating to the status or operation of the remote IMD 16 (e.g., power status, communication mode status, error correction information, etc.), as well as other information relating to the operation of the remote IMD 16.

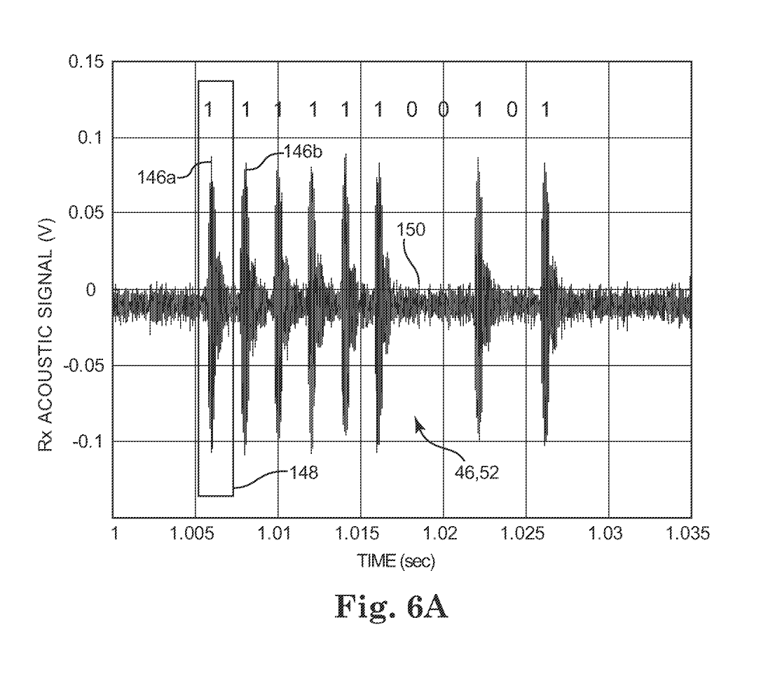

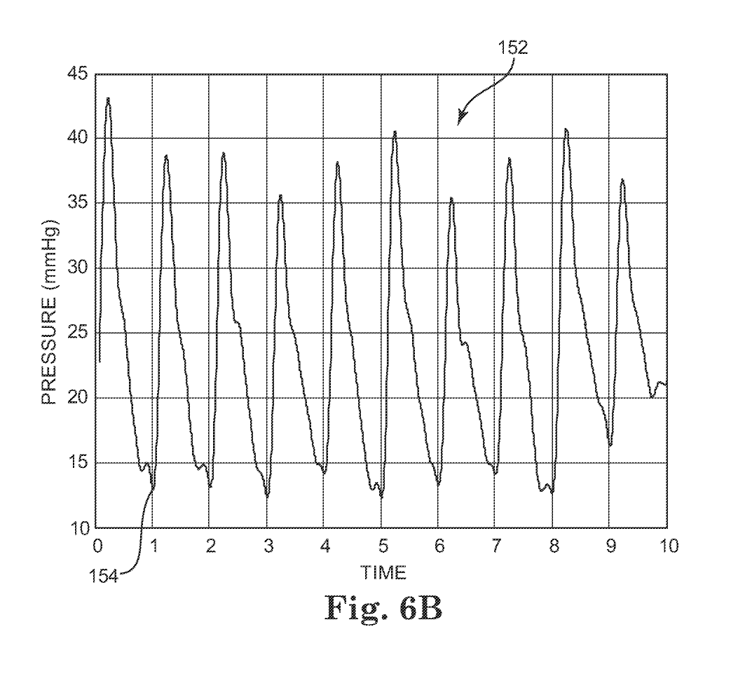

The sensor data obtained by the physiologic sensor 58 and transmitted via the ultrasound signal 46,52 may be encoded via on-off keying, phase-shift keying, frequency-shift keying, amplitude-shift keying, pulse code modulation, frequency modulation, amplitude modulation, or other suitable modulation technique used in telemetry protocols. In on-off keying, for example, digitized sensor data is transmitted acoustically within a modulated carrier ultrasound signal 46,52. The presence or absence of the carrier ultrasound signal 46,52 is detected by the external monitor 12 or pulse generator 14 as either a binary “1” or “0,” respectively. An example pressure waveform employing on-off keying modulation as part of the outbound ultrasound signal 46,52 is described further herein with respect to FIGS. 6A-6B.

The signal detector 62 is configured to generate an activation trigger signal to activate the remote IMD 16 via the activation/deactivation switch component 64. The activation trigger signal is generated by the signal detector 62 when the electrical signal generated by the ultrasonic transducer 42 exceeds a specific voltage threshold.

In response to the generation of the activation trigger signal by the signal detector 62, the switch component 64 is actuated to allow current to flow from the energy storage device 56 to the controller module 68, thereby placing the remote IMD 16 in the active state. The switch component 64 can also be actuated to prevent current from flowing to the controller module 68, thereby placing the remote IMD 16 in the standby or sleep state. Further details regarding the general construction and function of acoustic switches are disclosed in U.S. Pat. No. 6,628,989, entitled “Acoustic Switch And Apparatus And Methods For Using Acoustic Switches Within The Body,” which is expressly incorporated herein by reference in its entirety for all purposes. In other embodiments, the remote IMD 16 can include an antenna or inductive coil that receives an RF or inductive signal from the external monitor 12 or pulse generator 14 to activate or deactivate the remote IMD 16 within the body.

The controller module 68 includes a processor 74 such as a microprocessor or microcontroller coupled to a memory unit 76 that includes operating instructions and/or software for the remote IMD 16. The memory unit 76 can include volatile memory and nonvolatile memory. In some embodiments, nonvolatile memory can store code that includes bootstrap functions and device recovery operations, such as microprocessor reset. The nonvolatile memory may also include calibration data and parameter data in some embodiments. The volatile memory can include diagnostic and/or microprocessor-executable code, operating parameters, status data, and/or other data.

The controller module 68 can also include an oscillator or other timing circuitry 78 which directs the timing of activities to be performed by the remote IMD 16 once awoken from its low-power or sleep state. For example, the timing circuitry 78 can be used for timing the physiologic measurements taken by the physiologic sensor 58 and to generate timing markers to be associated with those measurements. The timing circuitry 78 may also be used for modulating the ultrasound signal 46,52.

The controller module 68, including the processor 74, can be configured as a digital signal processor (DSP), a field programmable gate array (FPGA), an application specific integrated circuit (ASIC)-compatible device, and/or any other hardware components or software modules for processing, analyzing, storing data, and controlling the operation of the remote IMD 16. Processor 74 executes instructions stored in the memory 96 or in other components such as, for example, the physiologic sensor(s) 58 or therapy delivery module 70 and/or other components or modules that may be present. In general, processor 74 executes instructions that cause the processor 74 to control or facilitate the functions of the remote IMD 16 and/or components of the remote IMD 16.

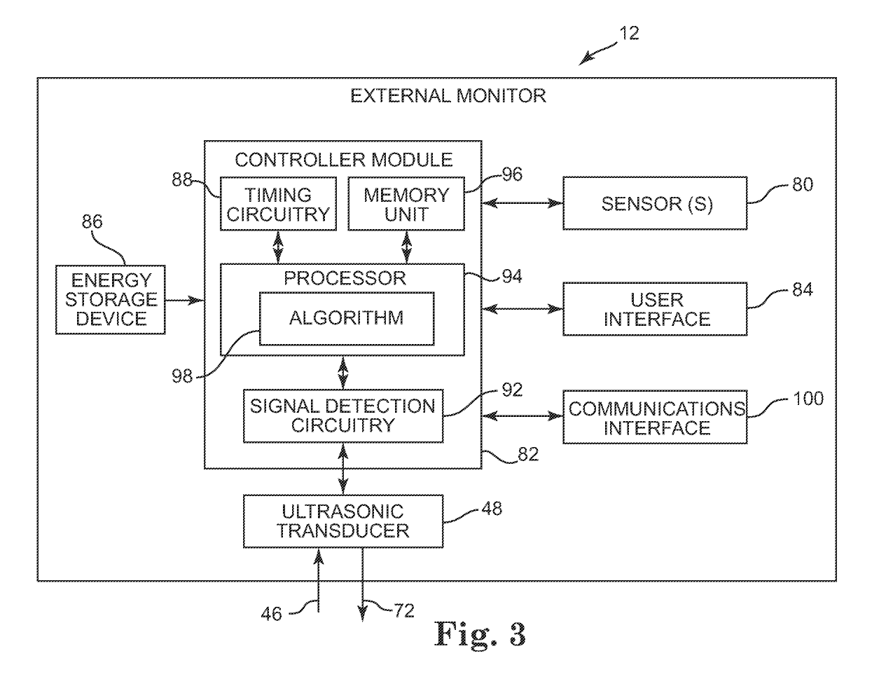

FIG. 3 is a block diagram showing several illustrative components of a communicating device such as the external monitor 12 of FIG. 1. As shown in FIG. 3, the external monitor 12 includes an ultrasonic transducer 48, one or more sensors 80, a controller module 82, a user interface 84, and an energy storage device 86. In some embodiments, the external monitor 12 is a handheld device. In other embodiments, the external monitor 12 is attached to a portion of the patient's body such as the patient's arm, neck, chest, thigh, or knee. The external monitor 12 can use any type of attachment mechanism, such as a strap, a patch, a belt, or any other means for coupling the monitor 12 to the patient's body.

The one or more sensors 80 can include a biosensor that generates a signal in response to a sensed physiologic parameter, or an environmental sensor that generates a signal in response to a sensed environmental parameter. In one embodiment, for example, the sensor 80 comprises a barometric pressure sensor configured to measure barometric pressure for use in calibrating pressure data sensed by the remote IMD 16. The external monitor 12 may include one or more additional sensors such as an ECG electrode sensor, a systemic blood pressure sensor, a posture sensor, a global positioning system (GPS) sensor, an activity sensor, a temperature sensor, a timer, and/or an oximeter.

The ultrasonic transducer 48 for the external monitor 12 can be configured to both transmit and receive ultrasound signals to and from the remote IMD 16. In other embodiments, the external monitor 12 includes at least one transducer configured for receiving ultrasound signals from the remote IMD 16 and at least one transducer configured for transmitting ultrasound signals to the remote IMD 16. The ultrasonic transducer 48 generates an electrical signal proportional to the magnitude of acoustic energy received by the transducer 48, which is then conveyed to the controller module 82 as an electrical waveform. In similar fashion, the ultrasonic transducer 48 generates an ultrasound signal proportional to the magnitude of the electrical energy generated by the controller module 82.

The controller module 82 includes circuitry for activating or controlling the sensor 80 and for receiving signals from the sensor 80. In some embodiments, the controller module 82 may include an oscillator or other timing circuitry 88 for use in modulating the ultrasound signal transmitted to the remote IMD 16 and/or the pulse generator 14 via the ultrasonic transducer 48. In some embodiments, the controller module 82 further includes signal detection circuitry 92 for detecting ultrasound signals 46 received from the remote IMD 16 and/or the pulse generator 14 via the ultrasonic transducer 48.

The controller module 82 includes a processor 94 for analyzing, interpreting, and/or processing the received ultrasound signal 46, and a memory unit 96 for storing the processed information and/or commands for use internally. The memory unit 96 can include volatile memory and nonvolatile memory. In some embodiments, nonvolatile memory can store code that includes bootstrap functions and device recovery operations, such as microprocessor reset. The nonvolatile memory may also include calibration data and parameter data in some embodiments. The volatile memory can include diagnostic and/or microprocessor-executable code, operating parameters, status data, and/or other data.

The controller module 82, including the processor 94, can be configured as a digital signal processor (DSP), a field programmable gate array (FPGA), an application specific integrated circuit (ASIC)-compatible device, and/or any other hardware components or software modules for processing, analyzing, storing data, and controlling the operation of the external monitor 12. Processor 94 executes instructions stored in the memory unit 96 or in other components such as, for example, the sensor(s) 80, user interface 84, communications interface 100 and/or other components or modules that may be present. In general, processor 94 executes instructions that cause the processor 94 to control or facilitate the functions of the external monitor 12 and/or components of the external monitor 12.

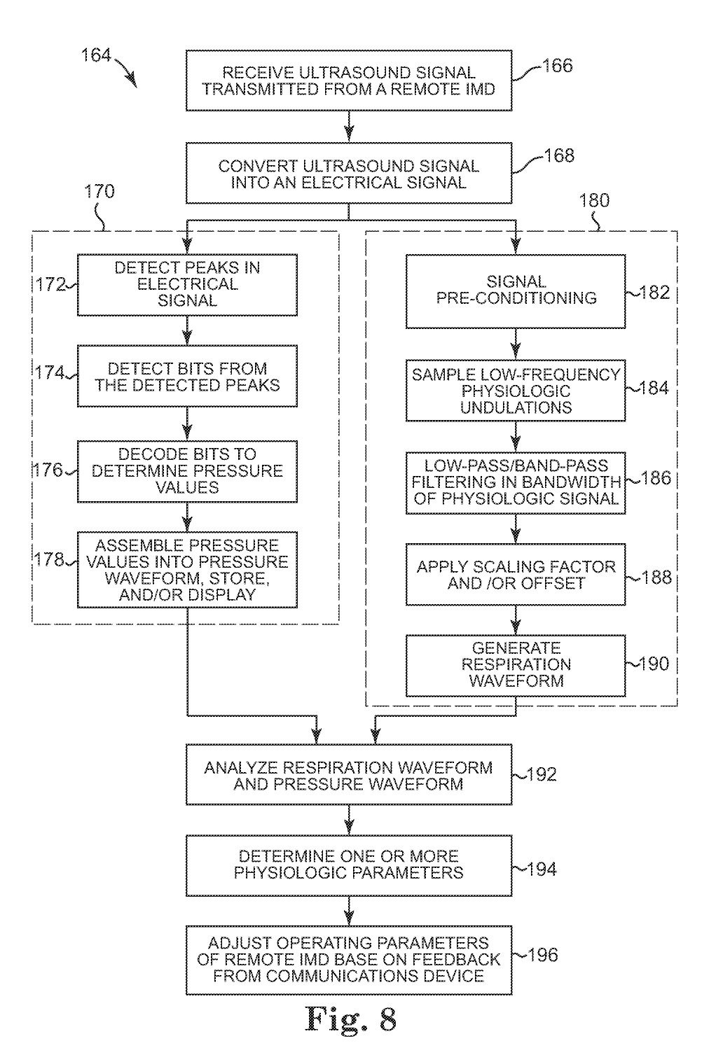

In certain embodiments, and as discussed further herein with respect to FIG. 8, the processor 94 can be configured to run an algorithm or routine 98 that, in addition to decoding the sensor data from the ultrasound signal 46 and analyzing the sensor data, also analyzes the amplitude and timing characteristics of the received ultrasound signal 46 to determine one or more additional physiologic parameters within the body based on a direct measure of the signal 46 itself. In one embodiment, for example, the amplitude and timing characteristics of the ultrasound signal 46 received by the external monitor 12 can be analyzed to determine a second physiologic waveform such as respiration, which can be correlated with the pressure waveform data encoded and transmitted as part of the ultrasound signal 46. The pressure and respiration waveforms can be further analyzed together to determine precisely the end diastolic pressure occurring at end expiration.

The user interface 84 can include a screen or display panel for communicating information to a physician and/or to the patient. In certain embodiments, the user interface 84 can also be used to display other information such as any physiologic parameters sensed by the remote IMD 16 or the external monitor 12 and the power and operational status of the remote IMD 16. The user interface 84 can also display information regarding the characteristics of the ultrasound signal 46 received from the remote IMD 16, including, but not limited to the pressure of the ultrasound signal 46, the carrier frequency of the ultrasound signal 46, and the modulation format of the ultrasound signal 46 (e.g., on-off keying, phase-shift keying, frequency-shift keying, amplitude-shift keying, pulse code modulation, frequency modulation, amplitude modulation, etc.), and/or the presence of any communication errors that may have occurred in the transmission.

In some embodiments, the external monitor 12 can include a communications interface 100 for connecting the monitor 12 to the Internet, an intranet connection, to a patient management database, and/or to other wired or wireless means for downloading and/or uploading information and programs, debugging data, and upgrades. According to some embodiments, the external monitor 12 is capable of operating in two modes: a user mode that provides useful clinical information to the patient or a caregiver, and a diagnostic mode that provides information to an individual for calibrating and/or servicing the external monitor 12 or for changing one or more parameters of the remote IMD 16.

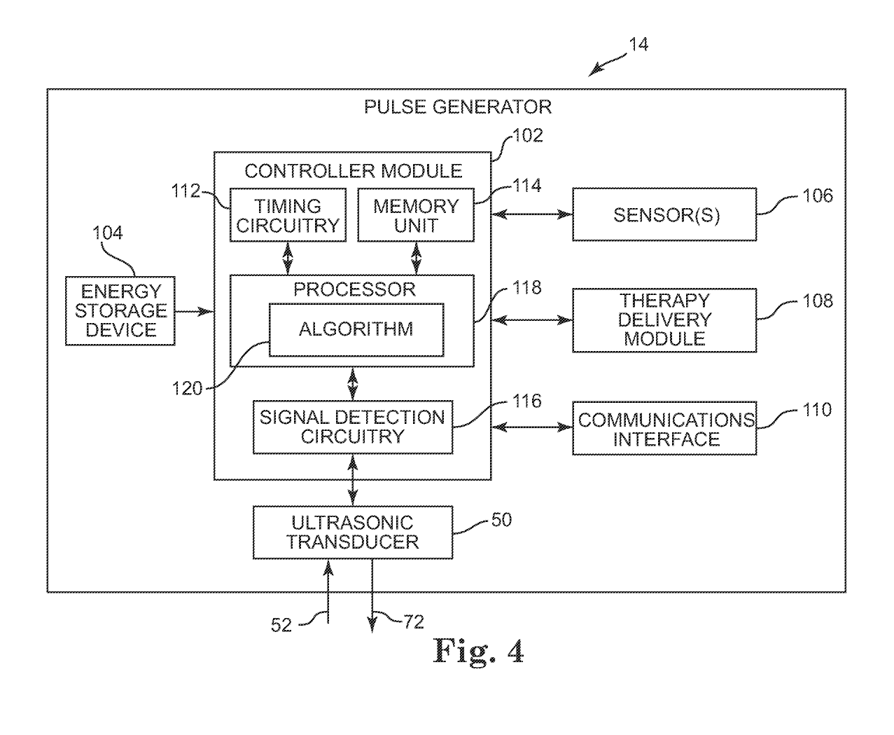

FIG. 4 is a block diagram showing several illustrative components of the pulse generator 14 of FIG. 1. As shown in FIG. 4, the pulse generator 14 includes an ultrasonic transducer 50, a controller module 102, an energy storage device 104, one or more sensors 106, a therapy delivery module 108, and a communications interface 110.

The sensors 106 can be configured to sense various electrical, mechanical, and chemical parameters within the body. In some embodiments, for example, the sensors 106 can comprise an electrode on a lead 36 coupled to the pulse generator 14 that can be used to measure various electrical parameters in or near the heart 18. The sensors 106 can also include an activity or motion sensor (e.g., an accelerometer) for detecting bodily movement, and a posture sensor for determining the patient's posture. The sensors 106 can also include a sensor for monitoring heart sounds and respiratory rhythms within the body. Other types of sensors 106 can also be used to sense other parameters within the body.

The therapy delivery module 108 can be utilized to provide therapy to the patient. In those embodiments in which the pulse generator 14 is a pacemaker or cardiac defibrillator, for example, the therapy delivery module 108 may provide electrical current to the lead 36 for pacing or shocking the heart 18. Alternatively, the therapy delivery module 108 may be utilized to provide other forms of therapy such as drug delivery.

A communications interface 110 allows communication between the pulse generator 14 and the external device 12, or between the pulse generator 14 and another device located inside or outside of the body. In certain embodiments, for example, the communications interface 110 includes an antenna or inductive coil that allows data, operational status, and/or other information to be transmitted back and forth between the pulse generator 14 and an external device. Alternatively, and in other embodiments, the communications interface 110 includes an ultrasonic transducer for acoustically communicating data, operational status, and other information to another device such as the external device 12.

The controller module 102 includes circuitry for controlling the sensor(s) 106, therapy delivery module 108, communications interface 110, as well as other components of the pulse generator 14. The controller module 102 further includes an oscillator, clock or other timing circuitry 112, and a memory unit 114. In some embodiments, the controller module 102 further includes signal detection circuitry 116 for detecting ultrasound signals 52 received from the remote IMD 16 via the acoustic transducer 50.

A processor 118 within the controller module 102 can be used to analyze, interpret, and/or process the received ultrasound signal 52. The controller module 102, including the processor 118, can be configured as a digital signal processor (DSP), a field programmable gate array (FPGA), an application specific integrated circuit (ASIC)-compatible device, and/or any other hardware components or software modules for processing, analyzing, storing data, and controlling the operation of the pulse generator 14. Processor 118 executes instructions stored in the memory unit 114 or in other components such as, for example, the sensor(s) 106, the therapy delivery module 108, the communications interface 100, and/or other components or modules that may be present. In general, processor 118 executes instructions that cause the processor 118 to control or facilitate the functions of the pulse generator 14 and/or components of the pulse generator 14.

In certain embodiments, and as discussed further with respect to FIG. 8, the processor 118 can be configured to run an algorithm or routine 120 that, in addition to, or in lieu of, analyzing the digitized sensor data generated by the remote IMD 16, also analyzes the amplitude and timing characteristics of the received ultrasound signal 52 to determine one or more additional physiologic parameters within the body based on a direct measure of the signal 52 itself. For example, in some embodiments the amplitude and timing characteristics of the ultrasound signal 52 can be analyzed to determine a second physiologic waveform such as respiration, which can be correlated with the pressure waveform data encoded and transmitted as part of the ultrasound signal 52. The pressure and respiration waveforms can be further analyzed to determine precisely the end diastolic pressure occurring at end expiration.

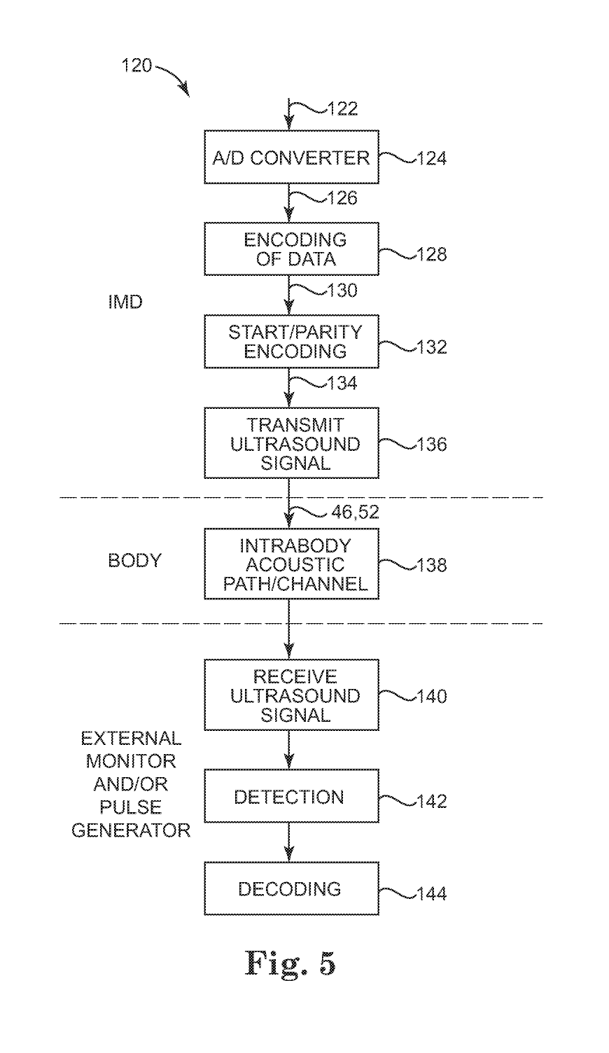

FIG. 5 is a diagram 120 showing several illustrative steps for sensing, sampling, encoding, and communicating a single pressure measurement through the body via the system 10 of FIG. 1. FIG. 5 may represent, for example, the sensing and communication of a single pressure measurement from a remote IMD 16 to a communicating device such as the external monitor 12 or pulse generator 14 shown in FIG. 1. As shown in FIG. 5, a pressure measurement 122 is measured with an analog to digital converter 124 (e.g., a 12 bit ADC), which converts the sensed pressure measurement 122 into a digitized format 126. If, for example, the pressure sensing element of the remote IMD 16 senses a pressure of 997.888 mmHg, and the ADC of the remote IMD 16 is 12 bits, corresponding to a resolution of the ADC equal to 0.125 mmHg in the 500-1011 mmHg pressure range, the ADC may output a digitized pressure 126 of 997.875 mmHg. The digitized pressure 126 is then encoded 128 using a suitable encoding protocol (e.g., on-off keying), producing an encoded data value 130. In some instances, the bandwidth or maximum data rate of the communication channel may be insufficient to support data transmission at the full resolution of the ADC. In such instances, the data encoded in the communication protocol may be reduced, for example, from 12 bits to 9 bits. By way of this example, the digitized pressure 126 with value 997.875 mmHg will become the encoded data value 130 equal to 998 mmHg. The digitized pressure measurement value of 998 mmHg, when encoded in this manner, may produce an encoded bit stream of “111110010.” In some embodiments, and as shown at block 132, the communication protocol may include additional encoding data such as a start bit (e.g., “1”) in the beginning of the bit stream and a parity bit (e.g., “1” or “0”) in the end of the bit stream, which can be utilized by a communicating device to determine the beginning of the bit stream and to detect the presence of any errors in the transmission. Although the additional encoding 132 may be performed as a separate step from the encoding of data, as shown in FIG. 5, in other embodiments both encoding steps 128,132 may be performed as a single step.

Once encoded, the remote IMD 16 may modulate the encoded data signal 134 and transmit 136 the data as an ultrasound signal 46,52 to the external monitor 12 or pulse generator 14. When initially transmitted from the ultrasound transducer 42, each of the bits in the ultrasound signal have the same amplitude and timing characteristics. As the ultrasound signal 46,52 propagates through the body from the remote IMD 16 to the external monitor 12 or pulse generator 14, as indicated generally at block 138, the amplitude and timing of each of the bits in the transmission are modulated slightly by the body due to time-varying changes in the patient's respiration, cardiac cycle, and patient movement. As a result, the amplitude and timing characteristics of the bits (i.e., “1”s) received 140 by the ultrasonic transducer 48,50 of the communicating device 12,14 are different from each other and those initially transmitted by the remote IMD 16. A digital or analog detection technique can then be used to detect 142 the peaks within the received ultrasound signal 46,52. The single pressure measurement (e.g., 998 mmHg) can then be decoded 144. The sensing, encoding, transmission, and decoding steps can then be repeated for each subsequent pressure value sensed by the remote IMD 16 and assembled into a pressure waveform representing the patient's blood pressure over the course of a measurement period.

FIGS. 6A-6B are several illustrative graphs showing the generation of a pressure waveform based on sensor data taken by the remote IMD 16 and transmitted via an ultrasound signal 46,52 to a communicating device such as the external monitor 12 or pulse generator 14 of FIG. 1. As shown in a first graph in FIG. 6A, the sensor data taken by the remote IMD 16 can be communicated using on-off keying, in which a binary “1” is represented in the acoustic waveform of the ultrasound signal 46,52 by the presence of an ultrasonic pulse 146a, shown bounded by time duration box 148. As can be further seen in FIG. 6A, the ultrasound signal 46,52 includes one pulse 146a,146b for each binary “1” in the encoded sensor data. Those portions of the ultrasound signal 46,52 in which a pulse is not present for a certain period of time (e.g., at point 150), in turn, each represent a binary “0” in the encoded sensor data.

FIG. 6B is a graph showing an illustrative pressure waveform generated by decoding the sensor data transmitted via the ultrasound signal 46,52. As shown over a period of 10 seconds in FIG. 6B, the encoded sensor data transmitted via the ultrasound signal 46,52 may be received and decoded by the external monitor 12 or the pulse generator 14 and converted into a pressure waveform 152. The encoded sensor data depicted generally in FIG. 6A may represent, for example, a single pressure data value occurring at any point 154 on the pressure waveform 152 in FIG. 6B.

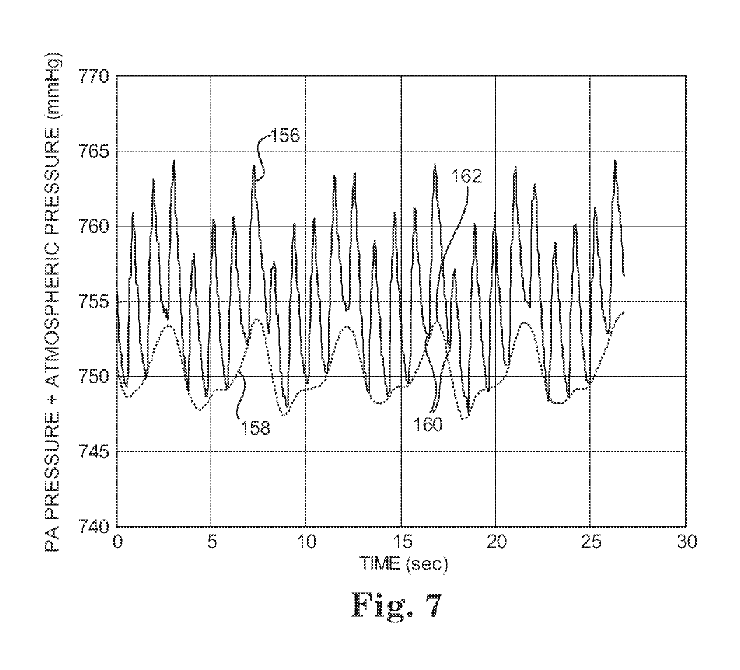

To obtain an accurate measurement of the end diastolic pressure (EDP) from the pressure waveform 152 in FIG. 6B, it is sometimes necessary to determine the end of the diastolic phase of the cardiac cycle occurring simultaneously with the expiration in the patient's respiration cycle. To accomplish this, some systems may attempt to derive a reference respiration signal directly from the pressure waveform 152 itself. As shown in the graph of FIG. 7, which represents an illustrative absolute (i.e., atmospheric plus gauge) pressure waveform 156 over a time period (T) of 30 seconds, one method to obtain a reference respiration waveform 158 may be by passing the waveform 156 through a low-pass filter and subtracting an offset pressure to generate a reference respiration waveform 158. The end diastolic pressure at end expiration may then be estimated by determining the end diastolic pressure from the pressure waveform 156 occurring at the end expiratory phase of the respiration waveform 158. This can be seen graphically, for example, where the local minimum pressure points 160 on the pressure waveform 156, representing minimum blood pressure at end diastole, correspond in time with the local maximum pressure points 162 on the respiration waveform 158, representing maximum intrathoracic pressure at end expiration, as shown.

In those systems in which the pressure waveform itself is used to derive the reference respiration waveform, the determination of end diastolic pressure at end expiration is vulnerable to pressure data loss caused, for example, by decoding errors in the acoustic communication, telemetry data dropout, measurement noise, and spurious events such as an arrhythmia, hiccups, and sudden motions. Additionally, the fidelity of the respiration waveform 158 is generally limited by the pressure waveform sampling frequency and amplitude quantization implemented in the remote IMD 16. If, for example, the sampling frequency of the pressure sensor is at 40 Hz for the illustrative pressure waveform 156 depicted in FIG. 7, then the time resolution of the reference respiration waveform 158 derived from the pressure waveform 156 is likewise 40 Hz. If, for example, the amplitude resolution of the IMD 16 is 1 mmHg and the pressure waveform 156 range is 20 mmHg, then the amplitude resolution of the reference respiration waveform 158 derived from the pressure waveform 156 is limited to 20 quantization levels. Such estimation techniques, therefore, are not always capable of providing an accurate measurement of end diastolic pressure at end expiration, particularly when the pressure waveform 156 has portions of the pressure data missing.

FIG. 8 is a flow chart showing an illustrative method 164 for determining one or more physiologic parameters within the body of a patient by analyzing the signal characteristics of an intrabody ultrasound signal 46,52 transmitted by the remote IMD 16 to a communicating device such as the external monitor 12 and/or pulse generator 14. In certain embodiments, for example, the method 164 may be performed by an algorithm or routine 98 of the external monitor 12 for determining end diastolic pressure at end expiration based on an analysis of the amplitude and timing characteristics of an ultrasound signal 46 transmitted by the remote IMD 16 to the external monitor 12. Alternatively, or in addition, the method 164 may be performed by an algorithm or routine 120 of the pulse generator 14 for determining end diastolic pressure at end expiration based on an analysis of the amplitude and timing characteristics of an ultrasound signal 52 transmitted by the remote IMD 16 to the pulse generator 14. In some embodiments, the method 164 may be performed by another device located inside or outside of the patient's body such as, for example, another remote IMD in acoustic communication with the remote IMD 16, or by the remote IMD 16 itself. Although the method 164 is described herein for use in deriving a respiratory waveform that can be used as a reference for determining end diastolic pressure of a pressure waveform, the method 164 may be used to derive other physiologic parameters within the body. Examples of other physiologic parameters that can be determined from an analysis of an intrabody ultrasound signal 46,52 include, but are not limited to, heart rate, respiratory rate, tidal volume, cardiac activity, patient movement, and patient posture.

The method 164 may begin generally at block 166 in which an ultrasound signal 46,52 is received for analysis. As can be understood further with respect to FIGS. 1 and 5, for example, block 166 may comprise the step of the external monitor 12 or pulse generator 14 receiving an ultrasound signal 46,52 transmitted from a remote IMD 16. In some embodiments, the remote IMD 16 may be prompted via a wake-up command sent from the external monitor 12 or pulse generator 14 to wake-up, take one or more sensor readings, and transmit an ultrasound signal 46,52 containing encoded sensor data. In other embodiments, the remote IMD 16 may be prompted by the external monitor 12 or pulse generator 14 to transmit an ultrasound signal 46,52 that does not contain any encoded sensor data. For example, the external device 12 or pulse generator 14 may prompt the remote IMD 16 to enter into an intermediate power state and activate only that circuitry required to transmit an ultrasound signal 46,52 for analysis back to the external monitor 12 or pulse generator 14 that does not contain any encoded sensor data.

From the received ultrasound signal 46,52, the external monitor 12 or pulse generator 14 may then convert the ultrasound signal 46,52 into a corresponding electrical signal (block 168). In those embodiments in which the electrical signal includes encoded pressure sensor data, the electrical signal may then be processed and decoded to extract the sensor data from the ultrasound signal 46,52 and generate a pressure waveform from the sensor data (block 170). As an example, the steps to decode the pressure data (block 170) may include a peak detection step (block 172) in which peaks in the ultrasound signal 46,52 are detected, and a bit detection step (block 174) in which binary “1”s and “0”s are determined from the detected peaks in the electrical signal. A decoding step (block 176) may then be used to determine pressure values from the bits. A pressure waveform is then assembled from the pressure values, stored, and/or displayed on a user interface (block 178). As discussed further herein, the pressure data obtained from the ultrasound signal 46,52 can then be combined with other physiologic parameter information obtained by an analysis of the characteristics of the ultrasound signal 46,52 itself.

As further shown in FIG. 8, the electrical signal generated (block 168) from the ultrasound signal 46,52 can be further analyzed (block 180) by the communicating device 12,14 to obtain one or more physiologic waveforms and parameters based on the amplitude and timing characteristics of the ultrasound signal 46,52 itself. A signal preconditioning step (block 182) may be applied to the ultrasound signal 46,52 prior to determining a physiologic waveform or parameter. At block 182, the algorithm or routine 98,120 can be configured to detect the relative peak of each acoustic pulse transmitted as part of the encoded sensor data in the received ultrasound signal 46,52. Detection of the peaks 126a,126b can be accomplished via any commonly known peak detection method, including fixed and variable threshold methods implemented in digital or analog circuitry. In one embodiment, peak detection may be accomplished using other signal preconditioning steps such as on-off keying demodulation, filtering, and pulse envelope detection.

After signal preconditioning (block 182), the signal may be sampled to provide a precursor to the physiologic waveform containing the low frequency undulations (block 184) created by physical modulation of the ultrasound signal 46,52 as it propagates through the body. For example, if the electrical signal (block 168) has been preconditioned by peak detection (block 182), extracting only the peaks will produce a precursor waveform having a variable sampling at approximately the bit transmission rate, such as, for example, 500 Hz. In a second example, the electrical signal (block 168) can instead be preconditioned by envelope detection (block 182) and sampled at a fixed rate higher than the bit transmission rate, producing an alternative precursor waveform that is evenly sampled.

Once conditioned and sampled, the resultant waveform may then be subjected to a low-pass or band-pass filtering step (block 186) with the filter bandwidth designed for the frequency range of the physiologic signal of interest. For example, a low pass filter with a 0.4 Hz cutoff frequency may be applied to extract respiratory oscillations from the precursor waveform and eliminate noise from the waveform. A 0.4 Hz cutoff frequency equates to twice a respiratory rate of 12 breaths per minute. A scaling factor and/or offset may then be applied (block 188) to each data point of the filtered waveform (block 186) to generate a respiration waveform (block 190) correlated in time with the pressure waveform generated at block 178.

An analysis (block 192) is then performed on both the respiration waveform generated at block 190 and/or the pressure waveform generated at block 178 in order to determine one or more physiologic parameters (block 194) in addition to the pressure waveform measured by the remote IMD 16. In those embodiments in which the sensor data comprises pressure data obtained from a remote IMD 16, for example, the respiration waveform generated at block 190 may be combined with a pressure waveform generated at block 178 in order to determine the end diastolic pressure at end expiration.

In some embodiments, and as further shown at block 196, the time at which end diastolic pressure at end expiration occurs, or another reference time point, can be used as feedback by the remote IMD 16 to trigger the IMD 16 to take sensor measurements during only a portion of the cardiac cycle. In some embodiments, for example, the timing of end diastolic pressure at end expiration can be used by the remote IMD 16 to gate the timing of the pressure measurements such that pressure data is taken only during the diastolic phase of the cardiac cycle, thus conserving power within the IMD 16.

In some embodiments, the respiration waveform can be used to determine other physiologic parameters and/or can be used as a reference to calibrate other implantable devices located within the body. In one embodiment, for example, an analysis of the respiration waveform can be used to derive respiration rate or tidal volume information, which can be used as an alternative to other sensors such as an accelerometer or an impedance-type respiration sensor, or to calibrate an accelerometer or impedance sensor implanted within the body. An analysis of the electrical signal generated from the ultrasound signal 46,52 can also be used to derive other physiological waveforms and determine other physiologic parameters within the body such as cardiac activity and/or physical motion. In some embodiments, for example, the electrical signal generated from the ultrasound signal 46,52 can be used to derive a cardiac waveform, which can be used to determine the presence of cardiac arrhythmia, extra beat or skipped beat, and aperiodic cardiac events, or can be used to determine other parameters such as heart rate.

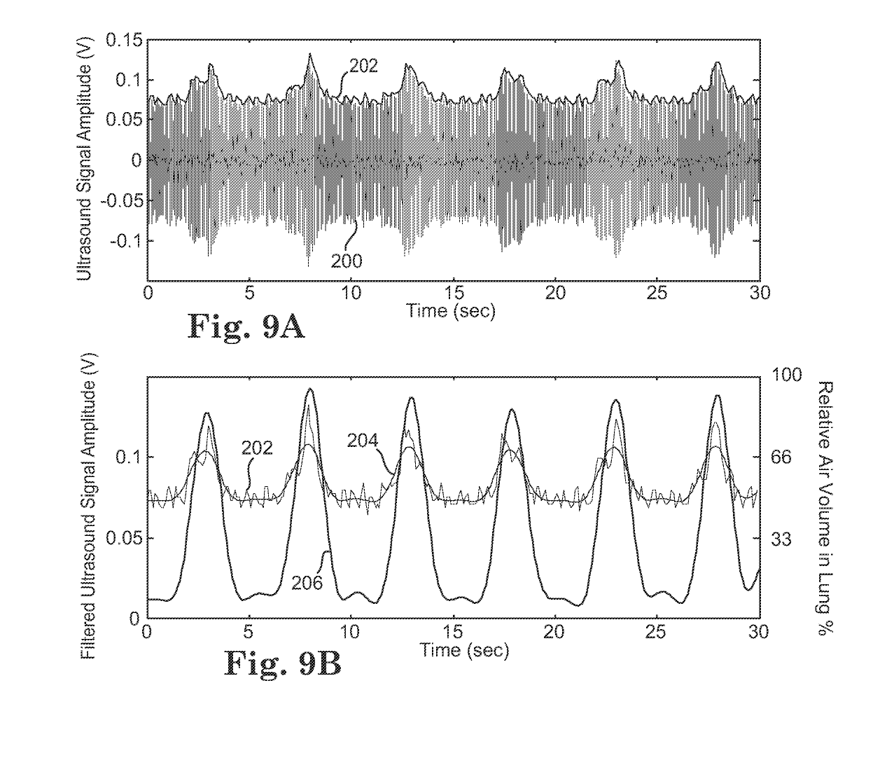

FIGS. 9A-9B show an illustrative respiration waveform 206 generated from an ultrasound signal 46,52 received by a communicating device such as the external monitor 12 or pulse generator 14. The respiration waveform 206 may represent, for example, a waveform generated by converting the ultrasound signal 46,52 into an electrical signal 200 and then passing the electrical signal through signal pre-conditioning and sampling circuitry, as discussed above, for example, with respect to blocks 182 and 184 in FIG. 8, resulting in waveform 202. As shown in FIG. 9A, electrical waveform 200 includes numerous peaks each of which are part of an acoustic bit of the encoded sensor data transmitted via the ultrasound signal 46,52 and shown in detail in FIG. 6A. The characteristics of waveform 202 can be further analyzed to determine one or more physiologic parameters in addition to, or in lieu of, the physiologic parameter(s) sensed by the remote IMD 16 and transmitted as part of the encoded sensor data within the ultrasound signal 46,52.

As can be further seen in FIG. 9B, the precursor waveform 202 resulting from steps 182 and 184 in FIG. 8 may be low-pass or band-pass filtered, as further discussed with respect to step 186, to extract a waveform 204 with a morphology indicative of the respiration waveform. A respiration waveform 206 representing relative lung inflation, for example, can be obtained by applying a scaling factor and offset to the filtered waveform 204 as in step 188 in FIG. 8.

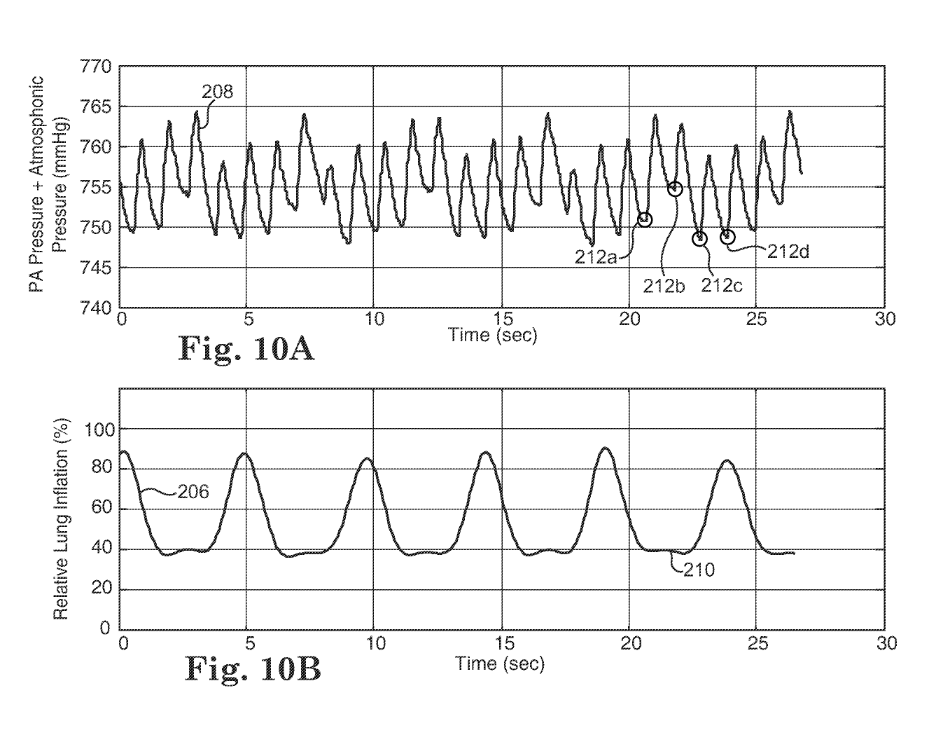

FIGS. 10A-10B show the determination of end diastolic pressure at end expiration from an illustrative pressure waveform 208 and the respiration waveform 206 of FIG. 9B. As can be further seen in FIG. 9B and in FIGS. 10A-10B, the respiration waveform 206 is inherently aligned in time with the pressure waveform 208 because it is derived from the electrical waveform 200 of the ultrasound signal 46,52 containing the encoded pressure data. On the respiration waveform 206, for example, end expiration 210 corresponding to the time at which lung inflation is at its lowest can be determined. Similarly, on the pressure waveform 208, end diastole corresponding to the end of the relaxation phase of the cardiac cycle can be determined. This can be seen at points 212a, 212b, 212c, and 212d on the pressure waveform 208 of FIG. 10A, which represent several end diastolic points corresponding to the end of the relaxation phase of the cardiac cycle. The end diastolic pressure at end expiration is then accurately determined by measuring the diastolic pressure 212b occurring at end expiration 210 of the respiration waveform 206.

Because the respiration waveform 206 is derived from the ultrasound signal 46,52 used to communicate the sensed pressure data instead of by analysis of the pressure sensor data, as discussed above with respect to FIG. 7, the respiration waveform 206 data is not subject to decoding errors such as decoding of the ultrasound signal 46,52. The time sampling resolution of the respiration waveform 206 is also not dependent on the time sampling frequency of the pressure sensor data sensed by the remote IMD 12. For an implantable pressure sensor configured to sample pressure at a sampling rate of 40 Hz and communicate pressure data at a data rate of 500 bits per second, for example, the resolution of the respiration waveform that can be derived from the ultrasonic signal 46,52 may minimally be the frequency of the communication (i.e., 500 Hz), which is much greater than the resolution of the pressure waveform sampling (i.e., 40 Hz).

The amplitude sampling resolution achieved by deriving the respiration waveform directly from the ultrasonic signal 46,52 is also greater in comparison to deriving the respiration waveform from the pressure waveform. The peak-to-peak amplitude range of the sensed pressure measurements is typically confined between a small range of pressure values. As can be seen in FIG. 7, for example, the amplitude range of a pulmonary artery pressure waveform 156 that includes atmospheric pressure may vary from between about 745 mmHg to 765 mmHg. For an operating range of 500 mmHg, a sampling rate of 40 Hz, and an encoding scheme incorporating error correction, within a fixed data throughput communication channel, the amplitude quantization may be reduced to no greater than ⅛ mmHg. The low resolution and low range of the actual pressure signal is typically insufficient to detect subtle changes in the respiration waveform when derived from the pressure waveform. In contrast, the voltage (V) variation in the electrical waveform obtained from the ultrasound signal 46,52 itself can be relatively large and finely quantized. As a result, the amplitude resolution of the respiration waveform derived from the ultrasound signal 46,52 itself is typically greater than that derived indirectly from the sensed pressure data.

Other characteristics in addition to the amplitude and timing of the ultrasonic pulses transmitted as part of the ultrasonic signal 46,52 can also be used to obtain useful information about the location and movement of the remote IMD 16 within the body, and the distance between the remote IMD 16 and the communicating device 12,14. If, for example, the remote IMD 16 is moving within the body relative to the communicating device (e.g., due to pulsitile blood flow within the pulmonary artery), the transmission of the ultrasound signal 46,52 will experience a frequency shift when transmitted through the body, which can be sensed by the communicating device 12,14 as a Doppler shift of the received signal 46,52. For example, if the transmission frequency of the ultrasound signal 46,52 is 40 KHz and the remote IMD 16 experiences a separation velocity of 1 m/s, the Doppler shift experienced by the communicating device 12,14 will be about 50 Hz. If the phase noise of the transmitted ultrasound signal 46,52 is relatively small, the Doppler shift can be obtained by recovering clock data from the remote IMD 16 and mixing it with the received ultrasound signal 46,52, similar to a homodyne receiver. The measured Doppler shift can then be used to analyze the relative motion of the remote IMD 16 to the communicating device 12,14 in the vector direction of the ultrasound signal 46,52.

In some embodiments, the transit time of the ultrasonic signal 46,52 between the remote IMD 16 and the communicating device 12,14 can be measured to determine the separation distance between the remote IMD 16 and the communicating device 12,14. For example, the period of each cycle of the ultrasound signal 46,52 can be measured, and the varying time period(s) caused by relative motion of the remote IMD and communicating device 12,14 can be measured to ascertain the separation distance between the two devices.

At relatively high transmission frequencies, the absorption of the ultrasonic signal 46,52 will tend to increase, and is largely dependent on the frequency of the transmission. The difference in attenuation of the ultrasound signal 46,52 at one frequency as compared to another frequency may thus provide a measure of the distance between the remote IMD 16 and the communicating device 12,14. Assuming, for example, an attenuation of about 1 dB per MHz per cm within the body, the attenuation of an ultrasound signal 46,52 transmitted at a first frequency (e.g., 5 MHz) to that of an ultrasound signal 46,52 transmitted at a second frequency (e.g., 10 MHz) could be used to detect relatively small translations of the remote IMD 16 within the body. Thus, by prompting the remote IMD 16 to transmit two ultrasound signals 46,52 each having a different frequency, the frequency-dependent absorption of each of the signals 46,52 can be used to measure the location of the remote IMD 16 within the body.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A method for determining one or more time-varying physiologic parameters within the body of a patient using intrabody ultrasound signals, comprising:
receiving an ultrasound signal transmitted from a remote device located within the body, the ultrasound signal including encoded sensor data measured by the remote device;
transducing the ultrasound signal into an electrical signal;
decoding the sensor data from the electrical signal and generating a first physiological waveform corresponding to the sensor data measured by the remote device; and
generating a second physiological waveform by analyzing fluctuations of the electrical signal caused by physiologic modulation of the ultrasound signal during propagation through the body.

2. The method of claim 1, further including analyzing at least one characteristic of the first and second physiological waveforms to determine one or more physiological parameters within the body.

3. The method of claim 1, wherein the first physiological waveform is a pressure waveform.

4. The method of claim 3, wherein the second physiological waveform is a respiration waveform.

5. The method of claim 3, wherein the second physiological waveform is a cardiac waveform.

6. The method of claim 1, further comprising using the one or more physiologic parameters to calibrate another device within the body.

7. The method of claim 1, wherein the remote device is a pressure sensor implanted within a pulmonary artery, and wherein the encoded sensor data comprises blood pressure data measured by the remote device within the pulmonary artery.

8. The method of claim 1, wherein generating a second physiological waveform includes filtering the electrical signal with a low-pass or band-pass filter having a bandwidth corresponding to the frequency range of a physiologic signal of interest.

9. The method of claim 2, wherein analyzing at least one characteristic of the first and second physiological waveforms to determine one or more physiologic parameters within the body includes detecting one or more peaks in the electrical signal and correlating the amplitude and timing of the peaks in the electrical signal with the measured sensor data from the first physiological waveform.

10. The method of claim 2, wherein analyzing at least one characteristic of the first and second physiological waveforms includes determining the end expiration stage of the patient's respiration cycle.

11. The method of claim 2, wherein analyzing at least one characteristic of the first and second physiological waveforms includes determining a respiration rate of the patient's respiration cycle.

12. The method of claim 2, wherein analyzing at least one characteristic of the first and second physiological waveforms includes determining a tidal volume of the patient's respiration cycle.

13. The method of claim 2, wherein analyzing at least one characteristic of the first and second physiological waveforms includes determining a heart rate.

14. The method of claim 2, wherein analyzing at least one characteristic of the first and second physiological waveforms includes determining the presence of at least one of a cardiac arrhythmia, extra beat or skipped beat, or aperiodic cardiac event.

15. The method of claim 1, further comprising adjusting at least one operating parameter of the remote device in response to the one or more physiologic parameters.

16. The method of claim 1, further comprising determining one or more device-related parameters of the remote device based at least in part on the amplitude, phase, and/or time delay of a carrier signal component of the received ultrasound signal.

17. The method of claim 16, wherein determining one or more device-related parameters of the remote device includes measuring a Doppler shift in the received ultrasonic signal.

18. The method of claim 16, further comprising prompting the remote device to transmit a first ultrasound signal at a first frequency and a second ultrasonic signal at a second frequency different than the first frequency, and wherein determining one or more device-related parameters includes measuring a separation distance between the remote device and a communicating device in acoustic communication with the remote device based on a measured change in attenuation of the first and second ultrasound signals received by the communicating device.

19. A method for determining one or more time-varying physiologic parameters within the body of a patient using intrabody ultrasound signals, comprising:
transmitting an ultrasound signal from a remote device located within the body to a communicating device in acoustic communication with the remote device;
receiving the ultrasound signal on an ultrasonic transducer of the communicating device and transducing the ultrasound signal into an electrical signal;
generating a physiological waveform by analyzing fluctuations of the electrical signal caused by physiologic modulation of the ultrasound signal during propagation through the body; and
analyzing the physiological waveform to determine one or more physiologic parameters within the body.

20. A system for determining one or more physiologic parameters within the body of a patient using an intrabody ultrasound signal, comprising;
a remote device including at least one ultrasound transducer adapted to transmit an intrabody ultrasound signal;
a communicating device in acoustic communication with the remote device, the communicating device including at least one ultrasound transducer configured to receive the ultrasound signal and transduce the ultrasound signal into an electrical signal; and
processing means for:
generating a physiological waveform by analyzing fluctuations of the electrical signal caused by physiologic modulation of the ultrasound signal during propagation through the body; and
analyzing at least one characteristic of the physiologic waveform to determine one or more physiologic parameters within the body.

21. The system of claim 20, wherein the physiological waveform is a respiration waveform.

22. The system of claim 20, wherein the physiological waveform is a cardiac waveform.

23. The system of claim 20, wherein the remote device is configured to measure blood pressure within a vessel of the body.

24. The system of claim 23, wherein the ultrasound signal includes encoded pressure data measured by the remote device, and wherein the processing means is further configured for decoding the pressure data from the ultrasound signal and generating a pressure wave corresponding to the pressure data measured by the remote device.

25. The system of claim 20, wherein the processing means is further configured for analyzing at least one characteristic of the physiologic waveform and at least one characteristic of the pressure waveform to determine one or more physiologic parameters within the body.
